(19) United States
(12) Patent Application Publication
(10) Pub. No.: US 2020/0174239 A1
(43) Pub. Date: Jun. 4, 2020

(54) CONTROL DEVICE FOR DENTAL MICROSCOPE

(71) Applicant: Tsung-Lung Hsieh, Kaohsiung City (TW)

(72) Inventor: Tsung-Lung Hsieh, Kaohsiung City (TW)

(21) Appl. No.: 16/682,088

(22) Filed: Nov. 13, 2019

(30) Foreign Application Priority Data
Nov. 30, 2018 (TW) 107216338

(51) Int. Cl. G02B 21/00; G02B 21/24; G02B 21/02; A61B 1/24; A61C 1/00; A61B 1/00

(52) U.S. Cl. CPC G02B 21/0012; G02B 21/241; G02B 21/025; A61B 1/00006; A61C 1/0023; A61B 1/00016; A61B 1/00039; A61B 1/24

(57) ABSTRACT

A control device for a dental microscope includes a control module and a driving module. The dental microscope includes an adjustment knob operable to adjust an operating parameter of the dental microscope. The control module includes at least one pedal and a signal transmitter coupled to the pedal. The signal transmitter generates and outputs a control signal upon the pedal being pressed. The driving module includes a motor connected to the adjustment knob, and a signal receiver configured to receive the control signal and to activate the motor to turn the adjustment knob according to the control signal.

### CROSS-REFERENCE TO RELATED APPLICATION

This application claims priority of Taiwanese Utility Model Patent Application No. 107216338, filed on Nov. 30, 2018.

### FIELD

The disclosure relates to an accessory for a dental microscope, and more particularly to a control device for adjusting an operating parameter of the dental microscope.

### BACKGROUND

Referring to FIG. 1, a conventional dental microscope 9 includes an objective lens assembly 91 and an adjustment knob 92 configured to control the objective lens assembly 91 to adjust focus and/or magnification of the conventional dental microscope 9. When a dentist is performing an intricate examination or a surgical operation on a patient using the conventional dental microscope 9, his/her hands are often busy doing other tasks and cannot be spared to adjust the conventional dental microscope 9. When the focus and/or magnification of the conventional dental microscope 9 must be adjusted, the examination or surgical operation may be interrupted since the dentist has to use at least one hand to operate the adjustment knob 92.

### SUMMARY

Therefore, an object of the disclosure is to provide a control device for a dental microscope that can alleviate at least one of the drawbacks of the prior art.

According to one aspect of the disclosure, the control device for a dental microscope includes a control module and a driving module. The dental microscope includes an adjustment knob operable to adjust an operating parameter of the dental microscope.

The control module includes at least one pedal, and a signal transmitter coupled to the at least one pedal. The signal transmitter is configured to generate and output a control signal upon the at least one pedal being pressed.

The driving module includes a motor to be connected to the adjustment knob, and a signal receiver in communication with the signal transmitter for receiving the control signal from the signal transmitter. The signal receiver is configured to activate the motor to turn the adjustment knob according to the control signal.

### BRIEF DESCRIPTION OF THE DRAWINGS

Other features and advantages of the disclosure will become apparent in the following detailed description of the embodiment(s) with reference to the accompanying drawings, of which:

FIG. 1 is a perspective view of a conventional dental microscope;

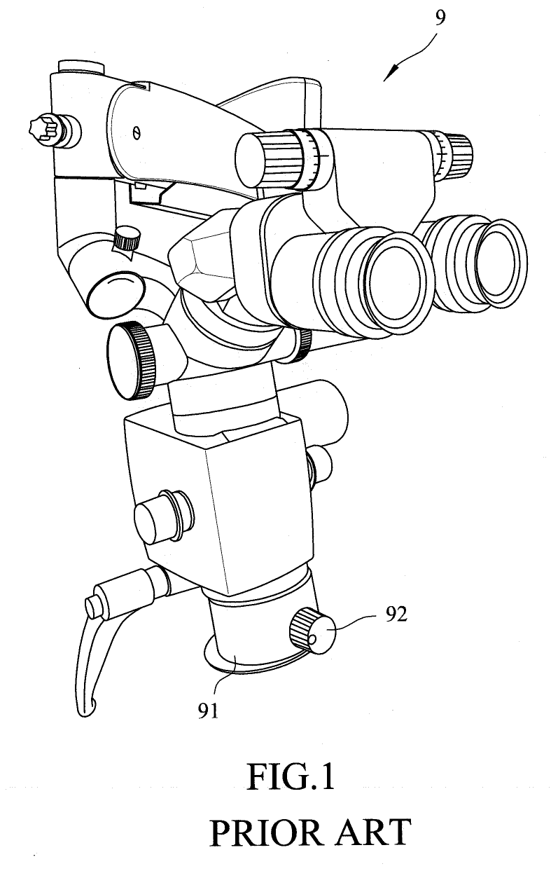

FIG. 2 is a schematic diagram of a control device for a dental microscope according to an embodiment of this disclosure;

FIG. 3 is a block diagram exemplarily illustrating components of the control device; and

FIG. 4 is a fragmentary perspective view illustrating a motor and a transmission mechanism of the control device according to this embodiment.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

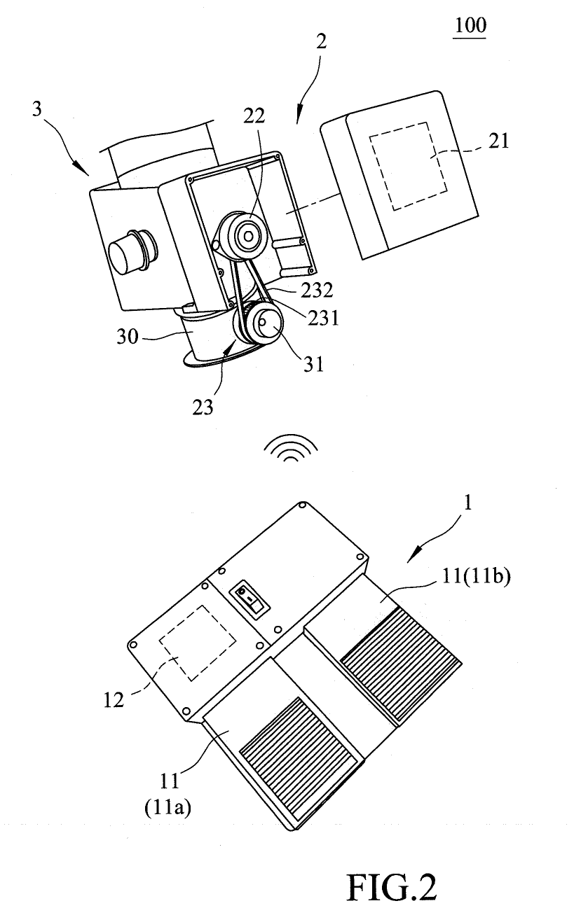

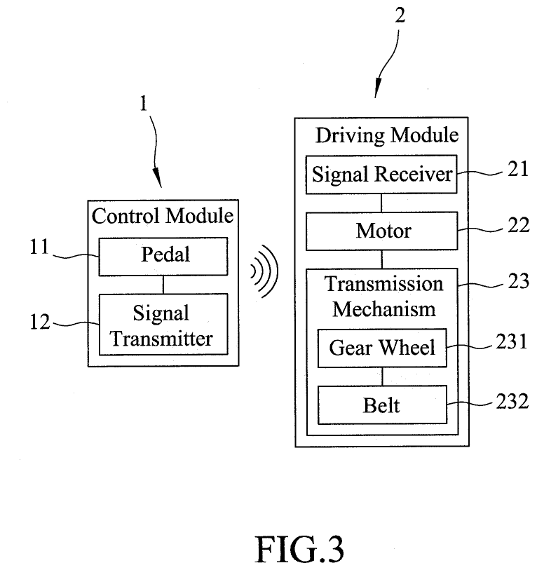

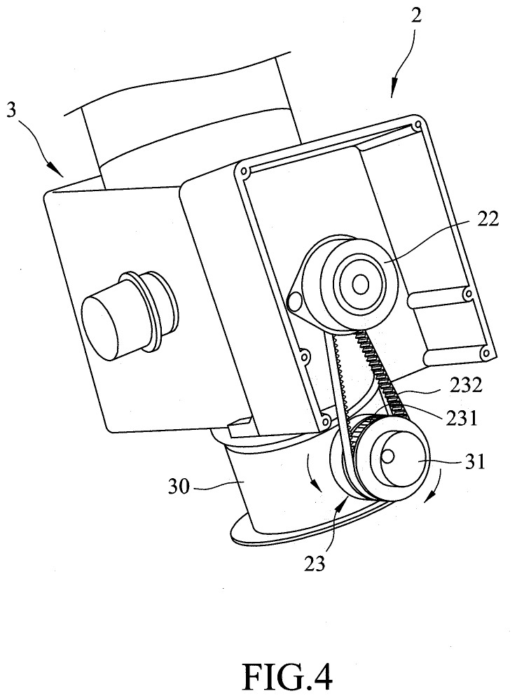

Referring to FIGS. 2 to 4, a control device 100 for a dental microscope 3 according to an embodiment of this disclosure is illustrated. The dental microscope 3 includes an objective lens assembly 30, and an adjustment knob 31 operable to adjust an operating parameter (e.g., focus or magnification) of the dental microscope 3 by adjusting elements (e.g., lenses, mirrors) of the objective lens assembly 30.

The control device 100 includes a control module 1 and a driving module 2. The control module 1 includes at least one pedal 11 to be placed on the floor near the dental microscope 3, and a signal transmitter 12 coupled to the at least one pedal 11. The signal transmitter 12 is configured to generate and output a control signal upon the at least one pedal 11 being pressed, for example, by a foot. In this embodiment, the signal transmitter 12 is electrically connected directly to the at least one pedal 11 through a wire, or wirelessly communicates with the at least one pedal 11.

In the embodiment illustrated in FIG. 2, the control module 1 includes two pedals 11a, 11b respectively on the left and right sides. The signal transmitter 12 is configured to generate a first control signal to serve as the control signal upon the pedal 11a on the left side being pressed, and to generate a second control signal to serve as the control signal upon the pedal 11b on the right side being pressed. In other embodiments, the control module 1 may include only one pedal 11, and the signal transmitter 12 may generate the first control signal upon a left part of the pedal 11 being pressed and generate the second control signal upon a right part of the pedal 11 being pressed.

The driving module 2 includes a signal receiver 21 in communication with the signal transmitter 12 for receiving the first and second control signals, a transmission mechanism 23, and a motor 22 connected to the adjustment knob 31 via the transmission mechanism 23. The transmission mechanism 23 includes a gearwheel 231 mounted coaxially on the adjustment knob 31, and a belt 232 connected to the motor 22 and trained on the gear wheel 231. The motor 22 is configured to drive the gear wheel 231 through the belt 232.

In this embodiment, the signal transmitter 12 is configured to communicate with the signal receiver 21 via wireless communication, e.g., Wi-Fi, Bluetooth®, infrared wireless communication, or the like. In other embodiments, the signal transmitter 12 may be electrically connected to the signal receiver 21 via physical wire(s) (not shown).

The signal receiver 21 is configured to activate the motor 22 to turn the adjustment knob 31 according to the control signal. Specifically, the signal receiver 21 is configured to activate the motor 22 to drive the gear wheel 231 through the belt 232 to turn the adjustment knob 31 counterclockwise upon receiving the first control signal, and to activate the motor 22 to drive the gear wheel 231 through the belt 232 to turn the adjustment knob 31 clockwise upon receiving the second control signal.

It should be noted that there is no particular limitation on the type of dental microscope that can make use of the control device 100 of this disclosure. Simply put, the control device 100 of this disclosure is suitable for use on any dental microscope that comes with an adjustment knob, including existing dental microscopes already in use by dental clinics or hospitals and existing models currently available on the market, and no adaption by re-configuring or re-structuring the dental microscope is necessary for utilizing the control device 100 of this disclosure to facilitate user operation of the dental microscope. When a dentist is using the dental microscope 3 equipped the control device 100 of this disclosure, he/she may adjust the focus and/or magnification of the dental microscope 3 by pressing the pedals 11a, 11b with his/her foot/feet without using his/her hand, and thus the examination or surgical operation will not be suspended.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A control device for a dental microscope, the dental microscope including an adjustment knob that is operable to adjust an operating parameter of the dental microscope, said control device comprising:
a control module including at least one pedal, and a signal transmitter coupled to said at least one pedal, said signal transmitter being configured to generate and output a control signal upon said at least one pedal being pressed; and
a driving module including
a motor to be connected to the adjustment knob, and
a signal receiver in communication with said signal transmitter for receiving the control signal from said signal transmitter, and configured to activate said motor to turn the adjustment knob according to the control signal.

2. The control device of claim 1, wherein said signal transmitter is configured to communicate with said signal receiver via wireless communication.

3. The control device of claim 1, wherein said driving module further includes a transmission mechanism including a gear wheel configured to be mounted coaxially on the adjustment knob, and a belt connected to said motor and trained on said gear wheel,
wherein said motor is configured to drive said gear wheel through said belt.

4. The control device of claim 1, wherein said at least one pedal includes two pedals.

5. The control device of claim 4, wherein said signal transmitter is configured to generate a first control signal to serve as the control signal upon one of said pedals being pressed, and to generate a second control signal to serve as the control signal upon the other one of said pedals being pressed,
wherein said signal receiver is configured to activate said motor to turn the adjustment knob counterclockwise upon receiving the first control signal, and to activate said motor to turn the adjustment knob clockwise upon receiving the second control signal.

6. The control device of claim 1, wherein said signal transmitter is configured to generate one of a first control signal and a second control signal to serve as the control signal, and said signal receiver is configured to activate said motor to turn the adjustment knob counterclockwise upon receiving the first control signal, and to activate said motor to turn the adjustment knob clockwise upon receiving the second control signal.
